(19) United States
(12) Patent Application Publication
(10) Pub. No.: US 2014/0194970 A1
(43) Pub. Date: Jul. 10, 2014

(54) GATE WIRE FOR CONTRALATERAL LEG ACCESS

(71) Applicant: TriVascular, Inc., Santa Rosa, CA (US)

(72) Inventor: Michael V. Chobotov, Santa Rosa, CA (US)

(73) Assignee: TriVascular, Inc., Santa Rosa, CA (US)

(21) Appl. No.: 14/151,373

(22) Filed: Jan. 9, 2014

Related U.S. Application Data
(60) Provisional application No. 61/750,851, filed on Jan. 10, 2013.

(51) Int. Cl. A61F 2/954; A61F 2/07

(52) U.S. Cl. CPC A61F 2/954; A61F 2/07
USPC 623/1.12

(57) ABSTRACT

An endovascular delivery system includes a pre-loaded, small guide wire for snaring via the contralateral side of a bifurcated prosthesis for deployment of a graft extension to the contralateral side of the bifurcated prosthesis. The pre-loaded guidewire avoids a cannulation step in the deployment of typical bifurcated stent grafts. Deployment methods using the pre-loaded guidewire and endovascular grafts having a pre-routed lumen for a secondary contralateral access guidewire are also described.

### CROSS-REFERENCE TO RELATED APPLICATIONS

This application claims the benefit of U.S. Provisional Application No. 61/750,851, filed Jan. 10, 2013, the contents of which are incorporated by reference herein.

### FIELD OF THE INVENTION

The present invention is related to an endovascular delivery system for an endovascular prosthesis. More particularly, the present invention is related to an endovascular delivery system for a bifurcated and inflatable prosthesis and a gate wire for contralateral leg access which avoids a separate cannulation procedure for accessing the contralateral leg during deployment of the prosthesis.

### BACKGROUND OF THE INVENTION

An aneurysm is a medical condition indicated generally by an expansion and weakening of the wall of an artery of a patient. Aneurysms can develop at various sites within a patient's body. Thoracic aortic aneurysms (TAAs) or abdominal aortic aneurysms (AAAs) are manifested by an expansion and weakening of the aorta. AAAs and TAAs are serious and life threatening conditions for which intervention is generally indicated. Existing methods of treating aneurysms include invasive surgical procedures with graft replacement of the affected vessel or body lumen or reinforcement of the vessel with a graft.

Surgical procedures to treat aortic aneurysms can have relatively high morbidity and mortality rates due to the risk factors inherent to surgical repair of this disease as well as long hospital stays and painful recoveries. This is especially true for surgical repair of TAAs, which is generally regarded as involving higher risk and more difficulty when compared to surgical repair of AAAs. An example of a surgical procedure involving repair of a AAA is described in a book titled Surgical Treatment of Aortic Aneurysms by Denton A. Cooley, M.D., published in 1986 by W.B. Saunders Company.

Due to the inherent risks and complexities of surgical repair of aortic aneurysms, endovascular aneurysm repair, or EVAR, has become a widely-used alternative therapy, most notably in treating AAAs. Early work in this field is exemplified by Lawrence, Jr. et al. in “Percutaneous Endovascular Graft Experimental Evaluation”, Radiology (May 1987) and by Mirich et al. in “Percutaneously Placed Endovascular Grafts for Aortic Aneurysms: Feasibility Study,” Radiology (March 1989). Commercially available endoprostheses for the endovascular treatment of AAAs include the Endurant™ and Talent™ Abdominal Stent Grafts sold by Medtronic, Inc. of Minneapolis, Minn.; the Zenith Flex® AAA Endovascular Graft and the Zenith TX2® TAA Endovascular Graft, both sold by Cook Medical, Inc. of Bloomington, Ind.; the AFX™ Endovascular AAA system sold by Endologix, Inc. of Irvine, Calif.; and the Gore® Excluder® AAA Endoprosthesis sold by W.L. Gore & Associates, Inc. of Flagstaff, Ariz. A commercially available stent graft for the treatment of TAAs is the Gore® TAG® Thoracic Endoprosthesis sold by W.L. Gore & Associates, Inc. of Flagstaff, Ariz.

When deploying devices by catheter or other suitable instrument, it is advantageous to have a flexible and low profile stent graft and delivery system for passage through the various guiding catheters as well as the patient's sometimes tortuous anatomy. Many of the existing endovascular devices and methods for treatment of aneurysms, while representing significant advancement over previous devices and methods, use systems having relatively large transverse profiles, often up to 24 French. Also, such existing systems have greater than desired lateral stiffness, which can complicate the delivery process. In addition, the sizing of stent grafts may be important to achieve a favorable clinical result. In order to properly size a stent graft, the treating facility typically must maintain a large and expensive inventory of stent grafts in order to accommodate the varied sizes of patient vessels due to varied patient sizes and vessel morphologies. Alternatively, intervention may be delayed while awaiting custom size stent grafts to be manufactured and sent to the treating facility. As such, minimally invasive endovascular treatment of aneurysms is not available for many patients that would benefit from such a procedure and can be more difficult to carry out for those patients for whom the procedure is indicated. Furthermore, in treating aneurysms near branched vessels multiple cannulation steps are often required to deploy stent grafts, including modular stent grafts, in the main and branched vessels. What have been needed are stent graft systems, delivery systems and methods that are adaptable to a wide range of patient anatomies and that can be safely and reliably deployed using a flexible low profile system.

### SUMMARY OF THE INVENTION

In one aspect of the present invention an endovascular system is provided. The endovascular delivery system may include a bifurcated and inflatable prosthesis include a main tubular body having an open end and opposed ipsilateral and contralateral legs defining a graft wall therein between, the graft wall having an inner graft wall surface and an outer graft wall surface, the ipsilateral and contralateral legs having open ends, and the main tubular body and the ipsilateral and contralateral legs optionally having inflatable channels; an elongate outer tubular sheath having an open lumen and opposed proximal and distal ends with a medial portion therein between, the proximal end of the outer tubular sheath securably disposed to a first handle at a handle assembly; an elongate inner tubular member having a tubular wall with an open lumen and opposed proximal and distal ends with a proximal portion near the proximal end, a distal portion near the distal end and a medial portion therein between, the inner tubular member having a longitudinal length greater than a longitudinal length of the outer tubular sheath, the inner tubular member being slidably disposed within the open lumen of the outer tubular sheath, the proximal end of the inner tubular member securably disposed to a second handle at the handle assembly; where the distal end of the outer tubular sheath being slidably disposed past and beyond the distal end of the inner tubular member to define a prosthesis delivery state and slidably retractable to the medial portion of the inner tubular member to define a prosthesis unsheathed state; a first elongate guidewire slidably disposed within the inner tubular member and extending from the handle assembly, through the ipsilateral leg of the prosthesis and through the main tubular body of the prosthesis in the prosthesis delivery state; a second elongate guidewire slidably disposed within the inner tubular member and having a proximal portion extending from the handle assembly or from a proximal portion of the inner tubular member, a medial portion extending through the ipsilateral leg of the prosthesis and a distal portion extending through at least a portion of the contralateral leg of the main tubular body of the prosthesis in the prosthesis delivery state; whereby the distal portion of the second elongate guidewire is engageable with a catheter for delivery of a contralateral graft extension within a portion of the contralateral leg of the main tubular body of the prosthesis in the prosthesis unsheathed state upon proximally retracting the second elongate guidewire. The distal portion of the second elongate guidewire may be disposed beyond the open end of the contralateral leg of the main tubular body in the prosthesis unsheathed state.

In an embodiment of the present invention, the endovascular delivery system may further include a delivery system second guidewire lumen extending over at least a portion of the medial portion of the second elongate guidewire and over at least a portion of the distal portion of the second elongate guidewire; where in the prosthesis delivery state the delivery system second guide wire lumen extends from at least a portion of the ipsilateral leg of the prosthesis to a portion of the contralateral leg of the prosthesis.

In an embodiment of the present invention, the endovascular delivery system may further include a third handle at the handle assembly; where the proximal portion of the second elongate guidewire is secured to the third handle to proximally retract the second elongate guidewire.

In an embodiment of the present invention, the endovascular delivery system may have the proximal portion of the second elongate guidewire extending through a wall of the proximal portion of the inner tubular member, where retraction of the outer tubular member engages the proximal portion of the second elongate guidewire to proximally retract the second elongate guidewire.

The prosthesis may include non-textile polymeric material, such as extruded polytetrafluoroethylene. The extruded polytetrafluoroethylene may be non-porous polytetrafluoroethylene. The prosthesis may further include a metallic expandable member securably disposed at or near the open end of the main tubular body of the prosthesis.

In another aspect of the present invention, A method for delivering a bifurcated prosthesis may include the steps of providing the endovascular delivery system according to the present invention; advancing the endovascular delivery system through a first branched artery and into an aneurysm in a main artery; retracting the outer sheath to deploy the prosthesis so the proximal end of the main tubular body of the prosthesis is disposed beyond the aneurysm and so that the ipsilateral and contralateral legs are disposed within the aneurysm; advancing a catheter through a second branched artery; engaging the catheter with the distal portion of the second elongate guidewire; retracting the second elongate guidewire proximally to advance the catheter within a portion of the contralateral leg of the prosthesis; disengaging the second elongate guidewire and the catheter from one and the other; and further retracting the second elongate guidewire through the ipsilateral leg of the prosthesis.

In one aspect of the present invention, the method may further include: maintaining the first elongate guidewire through the ipsilateral leg and the main tubular body of the prosthesis while retracting the second elongate guidewire through the ipsilateral leg of the prosthesis.

In some aspects of the present invention, the method of may further include: deploying a contralateral graft extension having a opposed proximal and distal open ends contained within the catheter so that the proximal end of the contralateral graft extension is disposed within a portion of the contralateral leg of the main tubular body of the prosthesis and so that the distal end of the contralateral graft extension is disposed distally of the aneurysm and within a portion of the second branched artery.

In other aspects of the present invention, the method may further include: advancing a second catheter through the first branched artery along the first elongate guidewire; deploying an ipsilateral graft extension having a opposed proximal and distal open ends contained within the second catheter so that the proximal end of the ipsilateral graft extension is disposed within a portion of the ipsilateral leg of the main tubular body of the prosthesis and so that the distal end of the ipsilateral graft extension is disposed distally of the aneurysm and within a portion of the first branched artery.

In another aspect of the present invention, a method for delivering a bifurcated prosthesis may include: providing the endovascular delivery system which may include a bifurcated and inflatable prosthesis including a main tubular body having an open end and opposed ipsilateral and contralateral legs defining a graft wall therein between; an elongate outer tubular sheath having an open lumen and opposed proximal and distal ends with a medial portion therein between; an elongate inner tubular member having a tubular wall with an open lumen and opposed proximal and distal ends with a proximal portion near the proximal end, a distal portion near the distal end and a medial portion therein between, the inner tubular member having a longitudinal length greater than a longitudinal length of the outer tubular sheath, the inner tubular member being slidably disposed within the open lumen of the outer tubular sheath, where the distal end of the outer tubular sheath being slidably disposed past and beyond the distal end of the inner tubular member to define a prosthesis delivery state and slidably retractable to the medial portion of the inner tubular member to define a prosthesis unsheathed state; a first elongate guidewire slidably disposed within the inner tubular member and extending through the ipsilateral leg of the prosthesis and through the main tubular body of the prosthesis in the prosthesis delivery state; a second elongate guidewire slidably disposed within the inner tubular member and extending through the ipsilateral leg of the prosthesis and having a distal portion extending through at least a portion of the contralateral leg of the main tubular body of the prosthesis in the prosthesis delivery state; advancing the endovascular delivery system through a first branched artery and into an aneurysm in a main artery; retracting the outer sheath to deploy the prosthesis so the proximal end of the main tubular body of the prosthesis is disposed beyond the aneurysm and so that the ipsilateral and contralateral legs are disposed within the aneurysm; engaging a second catheter with the distal portion of the second elongate guidewire (or with a third elongate guidewire that may have been exchanged for the second elongate guidewire); advancing the second catheter over the second elongate guidewire (or over a third elongate guidewire that may have been exchanged for the second elongate guidewire) through a second branched artery and; into a portion of the contralateral leg of the prosthesis; disengaging the second elongate guidewire and the catheter from one and the other; and further retracting the second elongate guidewire through the ipsilateral leg of the prosthesis.

In one aspect of the present invention, the method may further include maintaining the first elongate guidewire through the ipsilateral leg and the main tubular body of the prosthesis while retracting the second elongate guidewire through the ipsilateral leg of the prosthesis.

In another embodiment of the present invention an endovascular prosthesis may include a bifurcated and inflatable prosthesis which may have a main tubular body having an open end and opposed ipsilateral and contralateral legs defining a graft wall therein between, the graft wall having an inner graft wall surface and an outer graft wall surface, the ipsilateral and contralateral legs having open ends, and the main tubular body and the ipsilateral and contralateral legs having inflatable channels; and a prosthesis guidewire lumen extending along the inner lumen of the ipsilateral leg of the prosthesis and extending along at least a portion of the inner lumen of the contralateral leg of the prosthesis; where the prosthesis guidewire lumen is connected to or emanates from the distal end of the inner tubular member of the endovascular prosthesis delivery system, and is configured to receive a guidewire from a delivery system. The prosthesis guidewire lumen or extension thereof exits the contralateral leg opening and is releasably attached to the delivery system. The prosthesis guidewire lumen may include non-textile polymeric material, such as extruded polytetrafluoroethylene. The extruded polytetrafluoroethylene may be non-porous polytetrafluoroethylene.

In another aspect of the present invention, the method may further include deploying a contralateral graft extension having a opposed proximal and distal open ends contained within the catheter so that the proximal end of the contralateral graft extension is disposed within a portion of the contralateral leg of the main tubular body of the prosthesis and so that the distal end of the contralateral graft extension is disposed distally of the aneurysm and within a portion of the second branched artery.

In some aspects of the present invention, the method may further include advancing a second catheter through the first branched artery along the first elongate guidewire; deploying a ipsilateral graft extension having a opposed proximal and distal open ends contained within the second catheter so that the proximal end of the ipsilateral graft extension is disposed within a portion of the ipsilateral leg of the main tubular body of the prosthesis and so that the distal end of the ipsilateral graft extension is disposed distally of the aneurysm and within a portion of the first branched artery.

In further detail, as a replacement for the cannulation step in the deployment of typical bifurcated stent grafts, the present invention may use a pre-loaded, small (e.g. 0.014 inch or 0.018 inch coronary) guide wire for snaring via the contralateral side. The size of the wire may be governed by the need for visibility of its bright tip—as small as possible to adequately see and snare its tip. This wire may be pre-installed during assembly of the aortic body catheter. It may be routed from the handle, up the catheter shaft, into the ipsilateral leg, around the graft bifurcation, and out the contralateral leg before the graft is loaded into its catheter. It may project about 2 cm to about 4 cm distally from the contralateral leg (or whatever amount is required to easily snare), and have a bright floppy tip to facilitate visualization for snaring (and may also have a shaped tip to help in its snaring). Alternate embodiments could have more than one tip to enhance snaring. After the aortic body is filled with polymer, this gate wire or guidewire is snared from the contralateral side. The other end of the wire (at the handle) is able to be pulled (via a small knob or fitting crimped on the wire and exposed at the handle) to advance the snare up into the contra limb. As an alternative means of engaging and pulling the gate wire at the handle, the wire may project out the side of the hypotube and be engaged by the outer sheath knob in a secondary retraction. The wire is pulled about 6 cm to about 8 cm after it is snared to bring the end of the snare to the vicinity of the graft bifurcation. The snare is then released, and its loop is withdrawn from its catheter (while leaving its catheter in place). This catheter may be used to insert a 0.035 inch wire for contralateral iliac limb deployment, angiography, etc. A 0.014 inch wire would comprise on the order of only 1% of the cross sectional area of the catheter shaft, and would not traverse the section of highest material packing (the proximal stent and/or graft collar), so it is unlikely to have any measurable profile impact (larger wires can likely also be to be used without any discernible profile impact). This pre-routing of a gate wire eliminates the cannulation step (which could still be performed if the physician desires to do so), and also eliminates issues with advancing a cross over wire (or a wire from brachial access) into a bunched or bent contra leg, since the wire is pre-routed through the contra leg during catheter assembly. Such dimensions are non-limiting.

Another embodiment of the present invention may include a U-turn channel in the graft that allows the wire to be advanced around the graft bifurcation and further out the contralateral gate to facilitate snaring. This channel could be formed from, for example, polytetrafluoroethylene and attached to the inside of the graft body at the distal ends of each leg. The gate wire is pre-routed in this channel, and when the wire is pushed forward at the handle end, it would allow the wire to further extend out the contralateral leg opening to facilitate its capture by the snare. In addition, the end of the gate wire that projects from the catheter handle can be terminated by a knob or fitting that allows transmission of torque to the wire (such as a guide wire torquer), which would allow rotational repositioning of the wire tip to assist in its capture by the snare.

An alternative to using a pre-routed snare wire in the U-turn channel may be to permanently fix one end of the U-turn channel onto a lumen projecting from the distal end of the aortic body catheter. The lumen runs along the interior of the aortic body catheter and exits at the handle, where a port is located to allow introduction of a snare wire. The other end of the U-turn channel is held in place by the contralateral leg tether release wire. This would allow for insertion of a snare wire into the port of the catheter handle only when the operator desires to use a snare/crossover. When the snare wire is advanced into the catheter handle port, up the catheter shaft, around the inside of the deployed aortic body, and out the distal end of the U-turn channel, and finally out the end of the contra leg, the wire can be snared and a catheter used to gain access to the contralateral gate. The distal end of the U-turn channel would terminate a couple of cm short of the contra gate, while a tether connection would extend from its end to the third knob release wire. After gate access and contra limb deployment is performed, the third release knob is withdrawn to release not only the connection at the distal stop and the contra leg tether, but also the connection to the contralateral end of the U-turn channel. Thus, the U-turn channel is removed along with the aortic body catheter. In this embodiment, only the U-turn channel is pre-routed around the aortic body bifurcation, and not a guide wire.

These and other features and advantages of the present invention will become apparent from the following detailed description of illustrative embodiments thereof, which is to be read in connection with the accompanying drawings. Corresponding reference element numbers or characters indicate corresponding parts throughout the several views of the drawings.

### BRIEF DESCRIPTION OF THE DRAWINGS

FIG. 1 depicts an initial deployment state of the endovascular delivery system of the present invention within a patient's vasculature.

FIG. 2 depicts a deployment state of the endovascular delivery system of the present invention within a patient's vasculature after withdrawal of an outer sheath.

FIG. 3 depicts a deployment state of the endovascular delivery system of the present invention within a patient's vasculature after an initial and partial stent deployment.

FIG. 4 depicts a deployment state of the endovascular delivery system including a guidewire for contralateral leg access of the present invention within a patient's vasculature after a stent deployment.

FIG. 5 depicts a deployed bifurcated endovascular prosthesis with graft leg extensions.

FIG. 6 is a side elevational view of the endovascular delivery system of the present invention.

FIG. 7 is a side elevational and partial cutaway view of the proximal portion of the endovascular delivery system of the present invention.

FIG. 8 is a partial perspective and partial cutaway view of the proximal portion of the endovascular delivery system of the present invention.

FIG. 9 is an elevational view of the prosthesis of the present invention having a flap at the ipsilateral leg.

FIG. 10 is a partial elevational view of a distal stop on a delivery guidewire for restraining the ipsilateral leg of the prosthesis during certain delivery stages of the prosthesis.

FIG. 11 is an exploded and partial cut-away view of the distal stop initially engaging the ipsilateral leg flap.

FIG. 12 is an exploded and partial cut-away view of the distal stop engaging the ipsilateral leg flap.

FIG. 13 is an exploded view of a portion of the endovascular delivery system of FIG. 6 showing an embodiment for accessing a contralateral leg guidewire according to the present invention.

FIG. 14 is an exploded view of the proximal end of the contralateral leg guidewire of FIG. 13 guidewire according to the present invention.

FIG. 15 is an exploded view of the distal end of the contralateral leg guidewire of according to the present invention.

FIG. 16 depicts an endovascular graft of the present invention having an internal guidewire lumen according to the present invention.

FIG. 17 is a cross-section view of the ipsilateral leg of the endovascular graft of FIG. 16 taken along the 17-17 axis according to the present invention.

FIG. 18 depicts a portion of the endovascular delivery system of the present invention having a guidewire lumen disposed within portion of the ipsilateral and contralateral legs of the endovascular graft according to the present invention.

FIG. 19 depicts deployment of a graft leg extension without having to perform a separate cannulation step according to the present invention.

FIGS. 20 and 21 depict use of a magnetic snare according to the present invention.

FIGS. 22 and 23 depict interlocking magnets for snaring endovascular devices according to the present invention.

FIGS. 24 and 25 depict a funnel snaring device according to the present invention.

FIGS. 26 and 27 depict a catheter with a hooked and steerable lumen according to the present invention.

FIG. 28 depicts use of a detachable filament for contralateral leg access.

### DETAILED DESCRIPTION OF THE INVENTION

Embodiments of the invention are directed generally to methods and devices for treatment of fluid flow vessels with the body of a patient. Treatment of blood vessels is specifically indicated for some embodiments, and, more specifically, treatment of aneurysms, such as abdominal aortic aneurysms. With regard to graft embodiments discussed herein and components thereof, the term “proximal” refers to a location towards a patient's heart and the term “distal” refers to a location away from the patient's heart. With regard to delivery system catheters and components thereof discussed herein, the term “distal” refers to a location that is disposed away from an operator who is using the catheter and the term “proximal” refers to a location towards the operator.

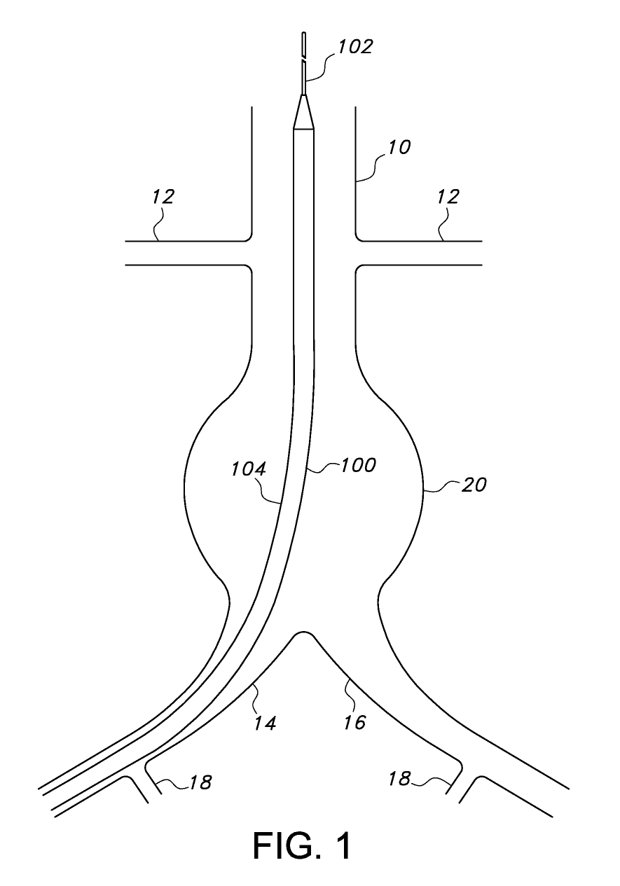

FIG. 1 illustrates an embodiment of a deployment sequence of an embodiment of an endovascular prosthesis (not shown), such as a modular stent graft assembly. For endovascular methods, access to a patient's vasculature may be achieved by performing an arteriotomy or cut down to the patient's femoral artery or by other common techniques, such as the percutaneous Seldinger technique. For such techniques, a delivery sheath (not shown) may be placed in communication with the interior of the patient's vessel such as the femoral artery with the use of a dilator and guidewire assembly. Once the delivery sheath is positioned, access to the patient's vasculature may be achieved through the delivery sheath which may optionally be sealed by a hemostasis valve or other suitable mechanism. For some procedures, it may be necessary to obtain access via a delivery sheath or other suitable means to both femoral arteries of a patient with the delivery sheaths directed upstream towards the patient's aorta. In some applications a delivery sheath may not be needed and the delivery catheter of the present invention may be directly inserted into the patient's access vessel by either arteriotomy or percutaneous puncture. Once the delivery sheath or sheaths have been properly positioned, an endovascular delivery catheter or system, typically containing an endovascular prosthesis such as but not limited to an inflatable stent-graft, may then be advanced over a guidewire through the delivery sheath and into the patient's vasculature.

FIG. 1 depicts the initial placement of the endovascular delivery system 100 of the present invention within a patient's vasculature. The endovascular delivery system 100 may be advanced along a first guidewire 102 proximally upstream of blood flow into the vasculature of the patient including iliac arteries 14, 16 and aorta 10 shown in FIG. 1. While the iliac arteries 14, 16 may be medically described as the right and left common iliac arteries, respectively, as used herein iliac artery 14 is described as an ipsilateral iliac artery and iliac artery 16 is described as a contralateral iliac artery. The flow of the patient's blood (not shown) is in a general downward direction in FIG. 1. Other vessels of the patient's vasculature shown in FIG. 1 include the renal arteries 12 and hypogastric arteries 18.

The endovascular delivery system 100 may be advanced into the aorta 10 of the patient until the endovascular prosthesis (not shown) is disposed substantially adjacent an aortic aneurysm 20 or other vascular defect to be treated. The portion of the endovascular delivery system 100 that is advanced through bodily lumens is in some embodiments a low profile delivery system; for example, having an overall outer diameter of less than 14 French. Other diameters are also useful, such as but not limited to less than 12 French, less than 10 French, or any sizes from 10 to 14 French or greater. Once the endovascular delivery system 100 is so positioned, an outer sheath 104 of the endovascular delivery system 100 may be retracted proximally so as to expose the prosthesis (not shown) which has been compressed and compacted to fit within the inner lumen of the outer sheath 104 of the endovascular delivery system 100.

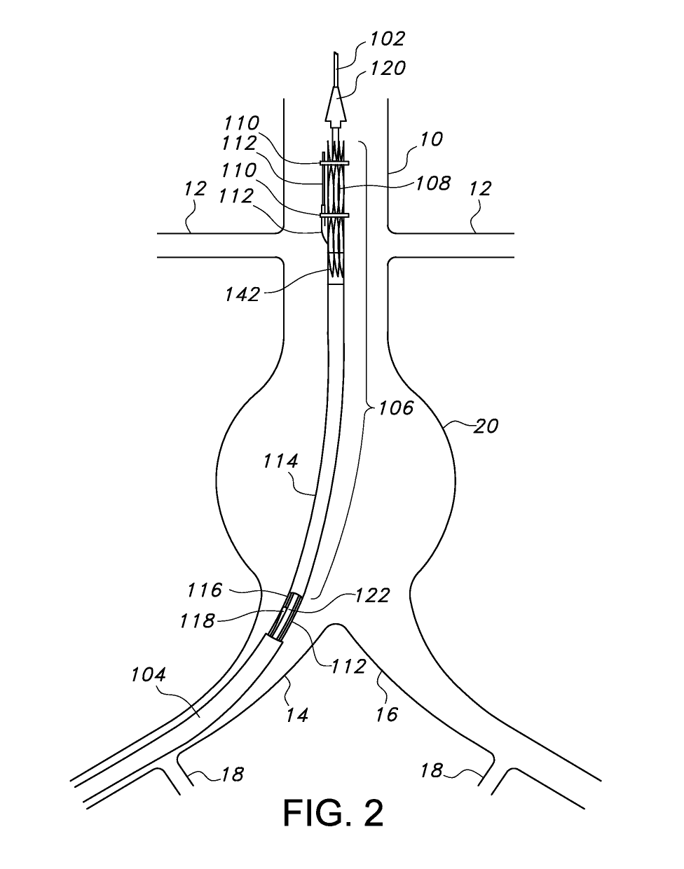

As depicted in FIG. 2, once the endovascular delivery system 100 is so positioned, the outer sheath 104 of the endovascular delivery system 100 may be retracted distally so as to expose the endovascular prosthesis 106 which has been compressed and compacted to fit within the inner lumen of the outer sheath 104 of the endovascular delivery system 100. The outer sheath 104 may be formed of a body compatible material. In some embodiments, the biocompatible material may be a biocompatible polymer. Examples of suitable biocompatible polymers may include, but are not limited to, polyolefins such as polyethylene (PE), high density polyethylene (HDPE) and polypropylene (PP), polyolefin copolymers and terpolymers, polytetrafluoroethylene (PTFE), polyethylene terephthalate (PET), polyesters, polyamides, polyurethanes, polyurethaneureas, polypropylene and, polycarbonates, polyvinyl acetate, thermoplastic elastomers including polyether-polyester block copolymers and polyamide/polyether/polyesters elastomers, polyvinyl chloride, polystyrene, polyacrylate, polymethacrylate, polyacrylonitrile, polyacrylamide, silicone resins, combinations and copolymers thereof, and the like. In some embodiments, the biocompatible polymers include polypropylene (PP), polytetrafluoroethylene (PTFE), polyethylene terephthalate (PET), high density polyethylene (HDPE), combinations and copolymers thereof, and the like. Useful coating materials may include any suitable biocompatible coating. Non-limiting examples of suitable coatings include polytetrafluoroethylene, silicone, hydrophilic materials, hydrogels, and the like. Useful hydrophilic coating materials may include, but are not limited to, alkylene glycols, alkoxy polyalkylene glycols such as methoxypolyethylene oxide, polyoxyalkylene glycols such as polyethylene oxide, polyethylene oxide/polypropylene oxide copolymers, polyalkylene oxide-modified polydimethylsiloxanes, polyphosphazenes, poly(2-ethyl-2-oxazoline), homopolymers and copolymers of (meth) acrylic acid, poly(acrylic acid), copolymers of maleic anhydride including copolymers of methylvinyl ether and maleic acid, pyrrolidones including poly(vinylpyrrolidone) homopolymers and copolymers of vinyl pyrrolidone, poly(vinylsulfonic acid), acryl amides including poly(N-alkylacrylamide), poly(vinyl alcohol), poly(ethyleneimine), polyamides, poly(carboxylic acids), methyl cellulose, carboxymethylcellulose, hydroxypropyl cellulose, polyvinylsulfonic acid, water soluble nylons, heparin, dextran, modified dextran, hydroxylated chitin, chondroitin sulphate, lecithin, hyaluranon, combinations and copolymers thereof, and the like. Non-limiting examples of suitable hydrogel coatings include polyethylene oxide and its copolymers, polyvinylpyrrolidone and its derivatives; hydroxyethylacrylates or hydroxyethyl(meth)acrylates; polyacrylic acids; polyacrylamides; polyethylene maleic anhydride, combinations and copolymers thereof, and the like. In some embodiments, the outer sheath 104 may be made of polymeric materials, e.g., polyimides, polyester elastomers (Hytrel®), or polyether block amides (Pebax®), polytetrafluoroethylene, and other thermoplastics and polymers. The outside diameter of the outer sheath 104 may range from about 0.1 inch to about 0.4 inch. The wall thickness of the outer sheath 104 may range from about 0.002 inch to about 0.015 inch. The outer sheath 104 may also include an outer hydrophilic coating. Further, the outer sheath 104 may include an internal braided or otherwise reinforced portion of either metallic or polymeric filaments. In addition to being radially compressed when disposed within an inner lumen of the outer sheath 104 of the endovascular delivery system 100, a proximal stent 108 may be radially restrained by high strength flexible belts 110 in order to maintain a small profile and avoid engagement of the proximal stent 108 with a body lumen wall until deployment of the proximal stent 108 is initiated. The belts 110 can be made from any high strength, resilient material that can accommodate the tensile requirements of the belt members and remain flexible after being set in a constraining configuration. Typically, belts 110 are made from solid ribbon or wire of a shape memory alloy such as nickel titanium or the like, although other metallic or polymeric materials are possible. Belts 110 may also be made of braided metal filaments or braided or solid filaments of high strength synthetic fibers such as Dacron®, Spectra or the like. An outside transverse cross section of the belts 110 may range from about 0.002 to about 0.012 inch, specifically, about 0.004 to about 0.007 inch. The cross sections of belts 110 may generally take on any shape, including rectangular (in the case of a ribbon), circular, elliptical, square, etc. The ends of the belts 110 may be secured by one or more stent release wires or elongate rods 112 which extend through looped ends (not shown) of the belts 110. The stent release wires or elongate rods 112 may be disposed generally within the prosthesis 106 during delivery of the system 100 to the desired bodily location. For example, the stent release wires or elongate rods 112 may enter and exit the guidewire lumen 122 or other delivery system lumen as desired to affect controlled release of the stent 108, including if desired controlled and staged release of the stent 108. Once the outer sheath 104 of the endovascular delivery system 100 has been retracted, the endovascular delivery system 100 and the endovascular prosthesis 106 may be carefully positioned in an axial direction such that the proximal stent 108 is disposed substantially even with the renal arteries.

In some embodiments, the endovascular prosthesis 106 includes an inflatable graft 114. The inflatable graft may be a bifurcated graft having a main graft body 124, an ipsilateral graft leg 126 and a contralateral graft leg 128. The inflatable graft 114 may further include a fill port 116 in fluid communication with an inflation tube 118 of the endovascular delivery system 100 for providing an inflation medium (not shown). The distal portion of the endovascular delivery system 100 may include a nosecone 120 which provides an atraumatic distal portion of the endovascular delivery system 100. The first guidewire 102 is slidably disposed within a guidewire lumen 122 of the endovascular delivery system 100.

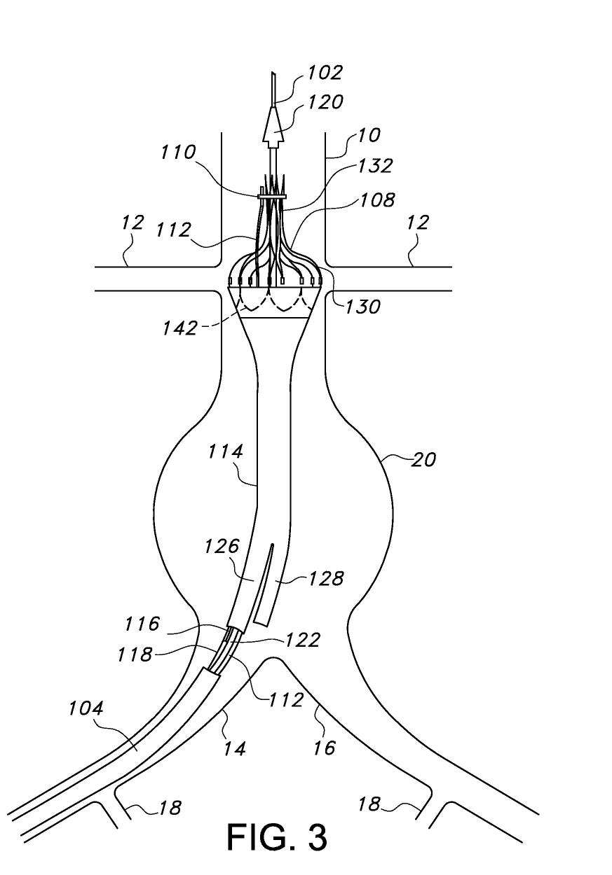

As depicted in FIG. 3, deployment of the proximal stent 108 may begin with deployment of the distal portion 130 of stent 108 by retracting the stent release wire or rod 112 that couples ends of belt 110 restraining the distal portion 130 of the stent 108. The distal portion 130 of stent 108 may be disposed to the main graft body 124 via a connector ring 142. The stent 108 and/or the connector ring 142 may be made from or include any biocompatible material, including metallic materials, such as but not limited to, nitinol (nickel titanium), cobalt-based alloy such as Elgiloy, platinum, gold, stainless steel, titanium, tantalum, niobium, and combinations thereof. The present invention, however, is not limited to the use of such a connector ring 142 and other shaped connectors for securing the distal portion 130 of the stent 108 at or near the end of the main graft body 124 may suitably be used. Additional axial positioning typically may be carried out even after deploying the distal portion 130 of the stent 108 as the distal portion 130 may provide only partial outward radial contact or frictional force on the inner lumen of the patient's vessel or aorta 10 until the proximal portion 132 of the stent 108 is deployed. Once the belt 110 constraining the proximal portion 132 of the stent 108 has been released, the proximal portion 132 of the stent 108 self-expands in an outward radial direction until an outside surface of the proximal portion 132 of the stent 108 makes contact with and engages an inner surface of the patient's vessel 10.

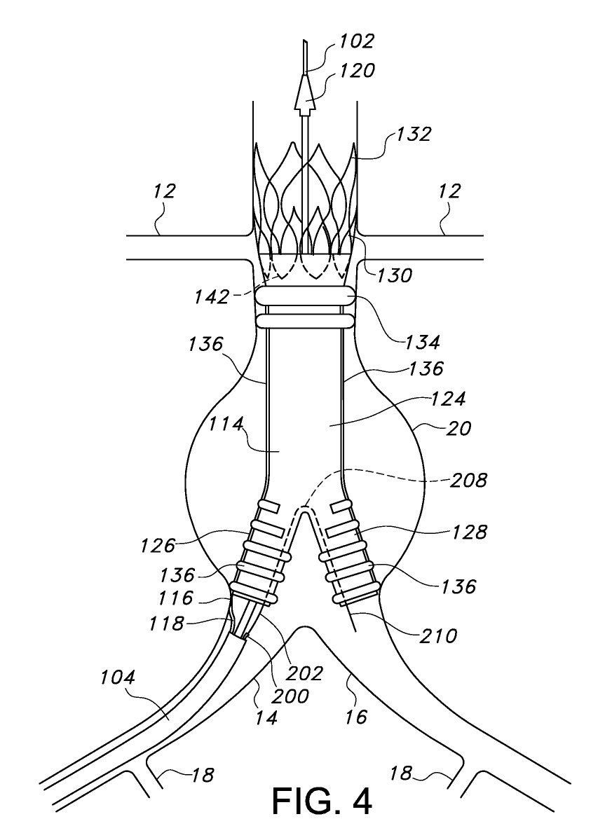

As depicted in FIG. 4, after the distal portion 130 of the stent 108 has been deployed, the proximal portion 132 of the stent 108 may then be deployed by retracting the wire 112 that couples the ends of the belt 110 restraining the proximal portion 132 of the stent 108. As the proximal portion 132 of the stent 108 self-expands in an outward radial direction, an outside surface of the proximal portion 132 of the stent 108 eventually makes contact with the inside surface of the patient's aorta 10. For embodiments that include tissue engaging barbs (not shown) on the proximal portion 132 of the stent 108, the barbs may also be oriented and pushed in a general outward direction so as to make contact and engage the inner surface tissue of the patient's vessel 10, which further secures the proximal stent 108 to the patient's vessel 10.

Further as depicted in FIG. 4, a second guidewire 202 is disposed from the endovascular delivery system 100 through the ipsilateral graft leg 126 and through the contralateral graft leg 128. The distal portion 210 of second guidewire 202 may extend beyond the end of the contralateral graft leg 128. The present invention, however, is not so limited and the distal portion 210 of second guidewire 202 may be disposed substantially flush with the end of the contralateral graft leg 128 or may be disposed slightly within the contralateral graft leg 128. The medial portion 208 of second guidewire 202 extends through the ipsilateral graft leg 126 and through at least a portion of the contralateral graft leg 128. A second guidewire lumen 200 may be contained within the outer sheath 104 of the endovascular delivery system 100 for routing the proximal portion 206 of second guidewire 202 to the handle assembly or to a proximal portion of the inner tubular member 150, as described below in further detail.

Once the proximal stent 108 has been partially or fully deployed, the proximal inflatable cuff 134 may then be filled through the inflation port 116 with inflation material injected through an inflation tube 118 of the endovascular delivery system 100 which may serve to seal an outside surface of the inflatable cuff 134 to the inside surface of the vessel 10. The remaining network of inflatable channels 136 may also be filled with pressurized inflation material at the same time which provides a more rigid frame like structure to the inflatable graft 114. For some embodiments, the inflation material may be a biocompatible, curable or hardenable material that may cured or hardened once the network of inflatable channels 136 are filled to a desired level of material or pressure within the network or after passage of a predetermined period of time. Some embodiments may also employ radiopaque inflation material to facilitate monitoring of the fill process and subsequent engagement of graft extensions (not shown). The material may be cured by any of the suitable methods discussed herein including time lapse, heat application, application of electromagnetic energy, ultrasonic energy application, chemical adding or mixing or the like. Some embodiments for the inflation material that may be used to provide outward pressure or a rigid structure from within the inflatable cuff 134 or network of inflatable channels 136 may include inflation materials formed from glycidyl ether and amine materials. Some inflation material embodiments may include an in situ formed hydrogel polymer having a first amount of diamine and a second amount of polyglycidyl ether wherein each of the amounts are present in a mammal or in a medical device, such as an inflatable graft, located in a mammal in an amount to produce an in situ formed hydrogel polymer that is biocompatible and has a cure time after mixing of about 10 seconds to about 30 minutes and wherein the volume of said hydrogel polymer swells less than 30 percent after curing and hydration. Some embodiments of the inflation material may include radiopaque material such as sodium iodide, potassium iodide, barium sulfate, Visipaque 320, Hypaque, Omnipaque 350, Hexabrix and the like. For some inflation material embodiments, the polyglycidyl ether may be selected from trimethylolpropane triglycidyl ether, sorbitol polyglycidyl ether, polyglycerol polyglycidyl ether, pentaerythritol polyglycidyl ether, diglycerol polyglycidyl ether, glycerol polyglycidyl ether, trimethylolpropane polyglycidyl ether, polyethylene glycol diglycidyl ether, resorcinol diglycidyl ether, glycidyl ester ether of p-hydroxy benzoic acid, neopentyl glycol diglycidyl ether, 1,6-hexanediol diglycidyl ether, bisphenol A (PO)₂ diglycidyl ether, hydroquinone diglycidyl ether, bisphenol S diglycidyl ether, terephthalic acid diglycidyl ester, and mixtures thereof. For some inflation material embodiments, the diamine may be selected from (poly)alkylene glycol having amino or alkylamino termini selected from the group consisting of polyethylene glycol (400) diamine, di-(3-aminopropyl) diethylene glycol r, polyoxypropylenediamine, polyetherdiamine, polyoxyethylenediamine, triethyleneglycol diamine and mixtures thereof. For some embodiments, the diamine may be hydrophilic and the polyglycidyl ether may be hydrophilic prior to curing. For some embodiments, the diamine may be hydrophilic and the polyglycidyl ether is hydrophobic prior to curing. For some embodiments, the diamine may be hydrophobic and the polyglycidyl ether may be hydrophilic prior to curing.

The network of inflatable channels 136 may be partially or fully inflated by injection of a suitable inflation material into the main fill port 116 to provide rigidity to the network of inflatable channels 136 and the graft 114. In addition, a seal is produced between the inflatable cuff 134 and the inside surface of the abdominal aorta 10. Although it is desirable to partially or fully inflate the network of inflatable channels 136 of the graft 114 at this stage of the deployment process, such inflation step optionally may be accomplished at a later stage if necessary.

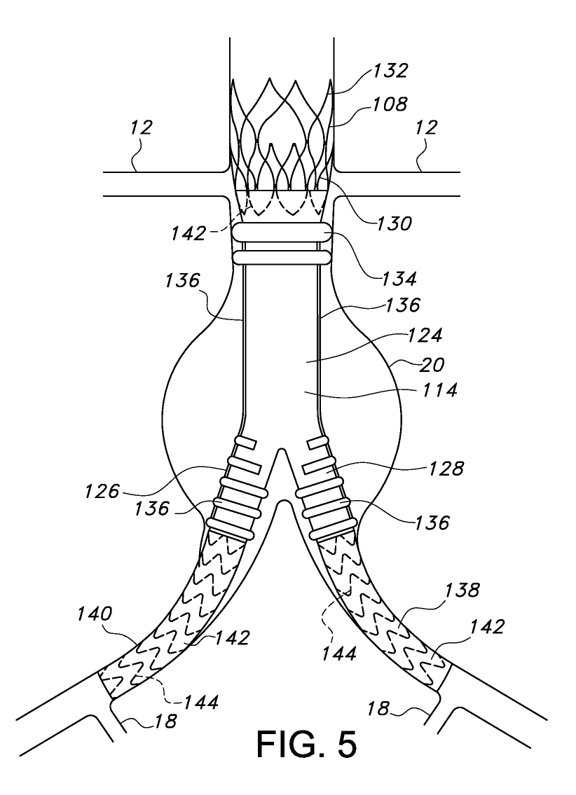

Once the graft 114 is deployed and the inflatable channels 136 thereof have been filled and expanded, another delivery catheter (not shown) may be used to deploy a contralateral graft extension 138, as depicted in FIG. 5. As described below, the catheter for delivering contralateral graft extension 138 is positioned to a location within the contralateral graft leg 128 through utilization of the second guidewire 202. By utilizing the second guidewire 202 for placement of the contralateral graft extension 138, a second, often difficult, cannulation step is avoided. Such a second cannulation step would involve deployment of a guidewire within the contralateral graft leg 128, and then utilizing that guidewire to deploy the catheter containing the contralateral graft extension 138.

Upon deployment, the contralateral graft extension 138 is in an axial position which overlaps the contralateral leg 128 of the graft 114. The amount of desired overlap of the graft extension 138 with the contralateral leg 128 may vary depending on a variety of factors including vessel morphology, degree of vascular disease, patient status and the like. However, for some embodiments, the amount of axial overlap between the contralateral graft extension 138 and the contralateral leg 128 may be about 1 cm to about 5 cm; more specifically, about 2 cm to about 4 cm. Once the contralateral graft extension 138 has been deployed, an ipsilateral graft extension 140 may be similarly deployed in the ipsilateral graft leg 126.

For some deployment embodiments, the patient's hypogastric arteries may be used to serve as a positioning reference point to ensure that the hypogastric arteries are not blocked by the deployment. Upon such a deployment, the distal end of a graft extension 138 or 140 may be deployed anywhere within a length of the ipsilateral leg 126 or contralateral leg 128 of the graft 114. Also, although only one graft extension 140, 138 is shown deployed on the ipsilateral side and contralateral side of the graft assembly 114, additional graft extensions 140, 138 may be deployed within the already deployed graft extensions 140, 138 in order to achieve a desired length extension of the ipsilateral leg 126 or contralateral leg 128. For some embodiments, about 1 to about 5 graft extensions 138, 140 may be deployed on either the ipsilateral or contralateral sides of the graft assembly 114. Successive graft extensions 138, 140 may be deployed within each other so as to longitudinally overlap fluid flow lumens of successive graft extensions.

Graft extensions 138, 140, which may be interchangeable for some embodiments, or any other suitable extension devices or portions of the main graft section 124 may include a variety of suitable configurations. For some embodiments, graft extensions 138, 140 may include a polytetrafluoroethylene (PTFE) graft 142 with helical nitinol stent 144.

Further details of the endovascular prosthesis 106 and/or graft extensions 138, 140 may be found in commonly owned U.S. Pat. Nos. 6,395,019; 7,081,129; 7,147,660; 7,147,661; 7,150,758; 7,615,071; 7,766,954 and 8,167,927 and commonly owned U.S. Published Application No. 2009/0099649, the contents of all of which are incorporated herein by reference in their entirety. Details for the manufacture of the endovascular prosthesis 106 may be found in commonly owned U.S. Pat. Nos. 6,776,604; 7,090,693; 7,125,464; 7,147,455; 7,678,217 and 7,682,475, the contents of all of which are incorporated herein by reference in their entirety. Useful inflation materials for the inflatable graft 114 may be found in may be found in commonly owned U.S. Published Application Nos. 2005/0158272 and 2006/0222596, the contents of all of which are incorporated herein by reference in their entirety. Additional details concerning delivery details, including systems, devices and methods, of the ipsilateral graft leg 126 and the contralateral leg 128 may be found in commonly owned U.S. Published Application No. 2013/0338760, the contents of which are incorporated the herein by reference in their entirety. Additional details of an endovascular delivery system having an improved radiopaque marker system for accurate prosthesis delivery may be found in commonly owned U.S. Published Application No. 2013/0338752, the contents of which are incorporated the herein by reference in their entirety.

Useful graft materials for the endovascular prosthesis 106 include, but are not limited, polyethylene; polypropylene; polyvinyl chloride; polytetrafluoroethylene (PTFE); fluorinated ethylene propylene; fluorinated ethylene propylene; polyvinyl acetate; polystyrene; poly(ethylene terephthalate); naphthalene dicarboxylate derivatives, such as polyethylene naphthalate, polybutylene naphthalate, polytrimethylene naphthalate and trimethylenediol naphthalate; polyurethane, polyurea; silicone rubbers; polyamides; polyimides; polycarbonates; polyaldehydes; polyether ether ketone; natural rubbers; polyester copolymers; silicone; styrene-butadiene copolymers; polyethers; such as fully or partially halogenated polyethers; and copolymers and combinations thereof. In some embodiments, the graft materials are non-textile graft materials, e.g., materials that are not woven, knitted, filament-spun, etc. that may be used with textile grafts. Such useful graft material may be extruded materials. Particularly useful materials include porous polytetrafluoroethylene without discernable node and fibril microstructure and (wet) stretched PTFE layer having low or substantially no fluid permeability that includes a closed cell microstructure having high density regions whose grain boundaries are directly interconnected to grain boundaries of adjacent high density regions and having substantially no node and fibril microstructure, and porous PTFE having no or substantially no fluid permeability. Such PTFE layers may lack distinct, parallel fibrils that interconnect adjacent nodes of ePTFE, typically have no discernable node and fibril microstructure when viewed at a magnification of up to 20,000. A porous PTFE layer having no or substantially no fluid permeability may have a Gurley Number of greater than about 12 hours, or up to a Gurley Number that is essentially infinite, or too high to measure, indicating no measurable fluid permeability. Some PTFE layers having substantially no fluid permeability may have a Gurley Number at 100 cc of air of greater than about 10⁶ seconds. The Gurley Number is determined by measuring the time necessary for a given volume of air, typically, 25 cc, 100 cc or 300 cc, to flow through a standard 1 square inch of material or film under a standard pressure, such as 12.4 cm column of water. Such testing may be carried out with a Gurley Densometer, made by Gurley Precision Instruments, Troy, N.Y. Details of such useful PTFE materials and methods for manufacture of the same may be found in commonly owned U.S. Patent Application Publication No. 2006/0233991, the contents of which are incorporated herein by reference in their entirety.

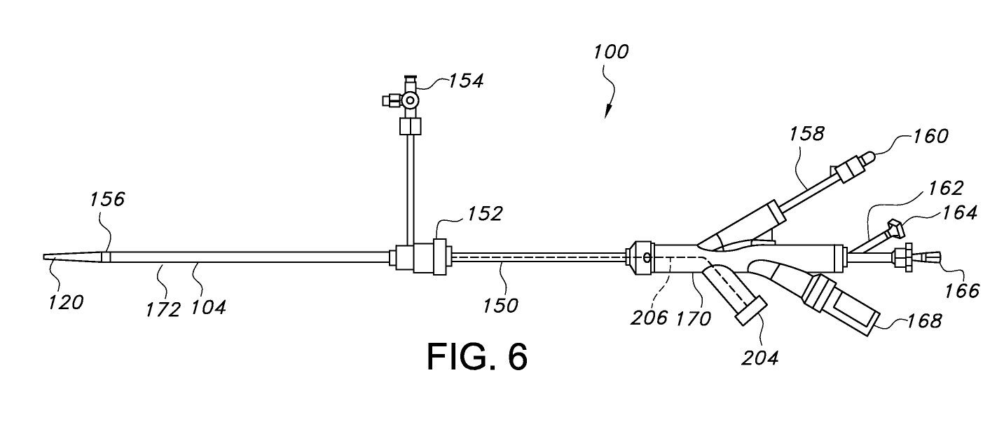

FIG. 6 is a side elevational view of the endovascular delivery system 100 of the present invention. The endovascular delivery system 100 may include, among other things, the nosecone 120; the outer sheath 104; a retraction knob or handle 152 for the outer sheath 104; a flush port 154 for the outer sheath 104; an outer sheath radiopaque marker band 156; an inner tubular member or hypotube 150; an inflation material or polymer fill connector port 158; an inflation material or polymer fill cap 160; a guidewire flush port 162; a guidewire flush port cap 164; a guidewire port 166; nested stent release knobs 168; and a second guidewire handle 204 engaged with the proximal portion 206 of second guidewire 202; interrelated as shown. The second guidewire handle 204, if desired, may be turned to torque the second guidewire 202 to rotationally control movement of the distal portion 210 of second guidewire 202. The second guidewire handle 204 is also useful for pulling the second guidewire 202 to retract the distal portion 210 of second guidewire 202 within a portion of the contralateral graft leg 128 of the endovascular prosthesis 106. The second guidewire handle 204 may also be used push the second guidewire 202 to advance the distal portion 210 of second guidewire 202. In such a case, for example, there may be slack distal portion 210 of second guidewire 202 engageable or otherwise associate with the second guidewire handle 204 to permit such advancement of the second guidewire 202. If the second guidewire is pre-loaded into the aortic body prosthesis in such a way that its bent medial portion 208 is proximal to the graft bifurcation by about 2-5 cm, then pulling the second guidewire handle 204 proximally will project the second guidewire end 210 a commensurate amount distally from the contralateral leg opening, as may be beneficial for snaring of the end 210 by an endovascular snare advanced from the contralateral iliac artery. Alternatively, advancing handle 204 distally would have the opposite effect, causing the end 210 to move proximally towards the contralateral leg opening.

The inner tubular member 150 may be formed from any of the above-described materials for the outer sheath 104. In addition, a portion of the inner tubular member 150 or even the entire inner tubular member 150 may be in the form of a metallic hypotube. Details of useful metallic hypotubes and endovascular delivery systems containing the same may be found in commonly owned U.S. Published Application No. 2013/0338753, the contents of which are incorporated herein by reference in their entirety.

The flush port 154 for the outer sheath 104 may be used to flush the outer sheath 104 during delivery stages. The outer sheath 104 may have a radiopaque marker band to aid the practitioner in properly navigating the delivery system 100 to the desired bodily site. The outer sheath 104 is retractable by movement of the retraction knob or handle 152 for the outer sheath 104 by a practitioner towards the proximal handle assembly 170 of the delivery system 100. The inner tubular member or hypotube 150 is disposed from the inner tubular member or hypotube 150 toward a proximal portion of the delivery system 100. The inflation material or polymer fill connector port 158 and the inflation material or polymer fill cap 160 are useful for providing inflation material (e.g., polymeric fill material) to inflate proximal inflatable cuffs 134 and the network of inflatable channels 136 of the inflatable graft 114. The guidewire flush port 162 and the guidewire flush port cap 164 are useful for flushing the guidewire port 166 during delivery stages of the delivery system 100. The nested stent release knobs 168 contains a series of nested knobs (not shown) that that are used to engage release mechanisms for delivery of the endovascular prosthesis 106. Further details, including but not limited to methods, catheters and systems, for deployment of endovascular prostheses are disclosed in commonly owned U.S. Pat. Nos. 6,761,733 and 6,733,521 and commonly owned U.S. Patent Application Publication Nos. 2006/0009833 and 2009/0099649, all of which are incorporated by reference herein in their entirety.

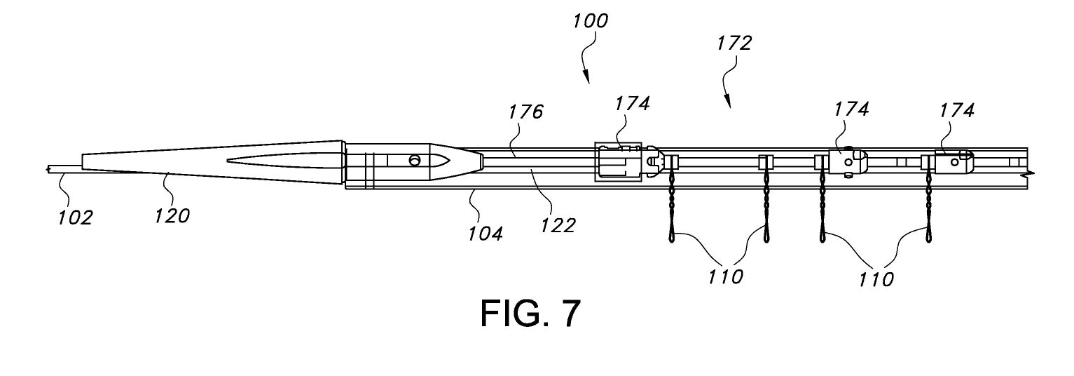

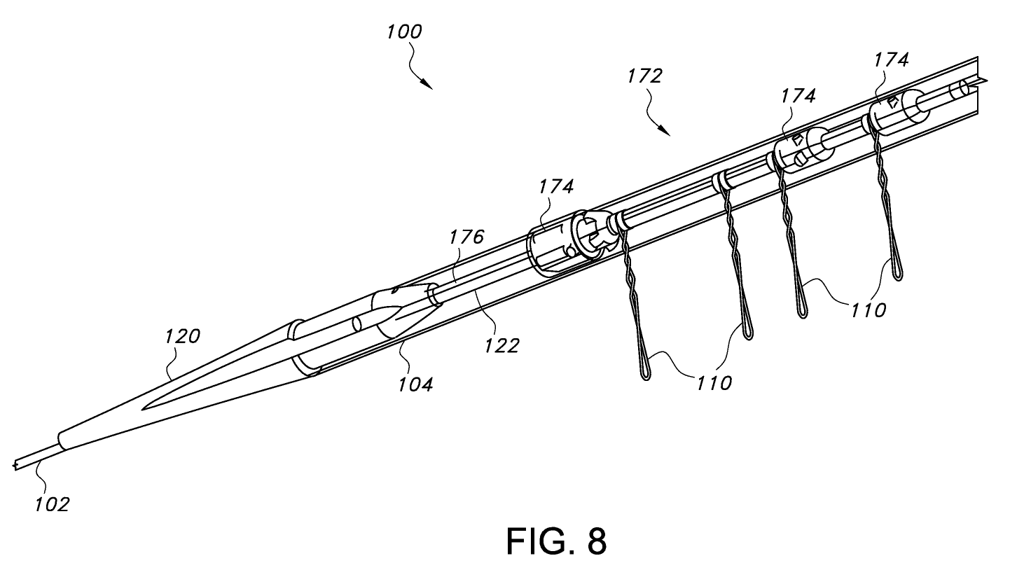

FIG. 7 is a side elevational and partial cutaway view of the proximal portion 172 of the endovascular delivery system 100 of the present invention, and FIG. 8 is a partial perspective and partial cutaway view of the proximal portion 172 of the endovascular delivery system 100 of the present invention. The proximal portion 172 of the endovascular delivery system 100 includes prosthesis/stent holders 174 disposed upon a prosthesis/stent holder guidewire 176. The holders 174 are useful releasably securing the endovascular prosthesis 106 (not shown) within the delivery system 100. The holders 174 inhibit or substantially inhibit undesirable longitudinal and/or circumferential movement of the endovascular prostheses 106 during delivery stages of the delivery system 100. Belts 110 serve to restrain the endovascular prosthesis 106 in a radially constrained stage until desired release of the endovascular prosthesis 106.

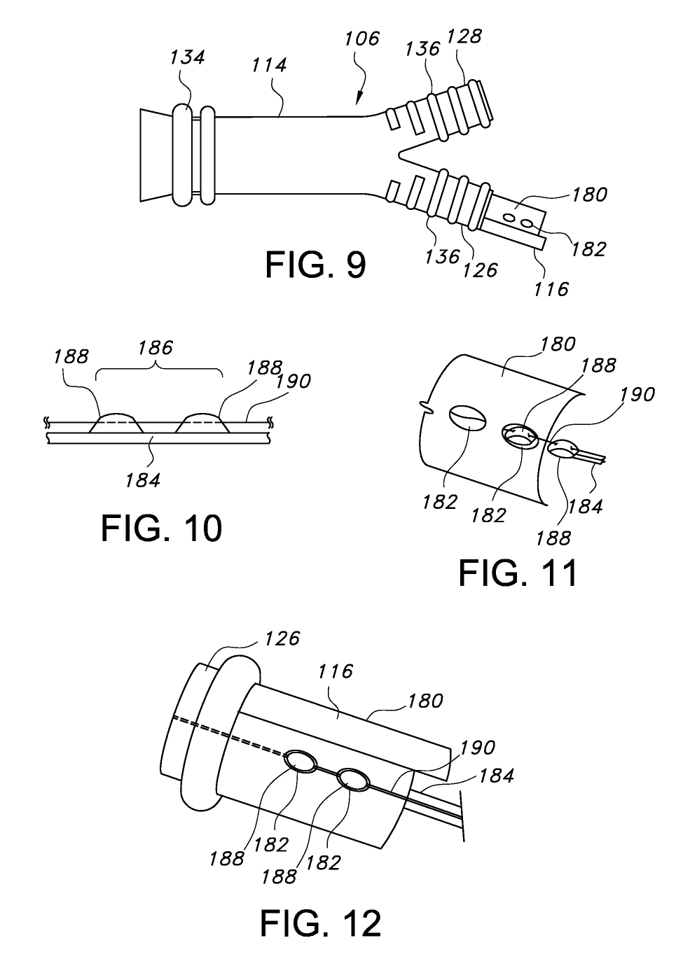

FIG. 9 is an elevational view of the prosthesis 106 of the present invention having a flap 180 at the ipsilateral leg 126. The flap 180 may be made from any of the above-described graft materials. In some embodiments, the flap 180 is made from polytetrafluoroethylene. The flap 180 may include two holes 182. The width of the flap may be from about 10% to about 90% of the circumference of the ipsilateral leg 126. In some embodiments, the width is from about 30% to about 60%; in other embodiments, from about 45% to about 55%. The flap 182 may contain two holes 182 as shown in FIG. 9, one hole, or more than two holes. A hole diameter of about 0.06 inches is useful, although hole diameters may be higher or lower. In the case of more than one hole, the hole diameters may vary between or among holes.

FIG. 10 is a partial elevational view of one embodiment including a distal stop 186 on a delivery guidewire 184 for restraining the ipsilateral leg 126 of the prosthesis 106 during certain delivery stages of the prosthesis 106. The distal stop 186 includes two raised projections 188 securably attached to a guidewire 184. A release wire 190 is slidably disposed within the projections 188. As depicted in FIGS. 11 and 12, the distal stop 186 is useful for releasably securing the ipsilateral leg 126, in particular the flap 180, to the distal stop 186 and the guidewire 184. The raised projections 188 may be secured or disposed within one or both of the flap holes 182. The release wire 190 is thus releasably inter-looped or inter-laced within or to the flap 180.

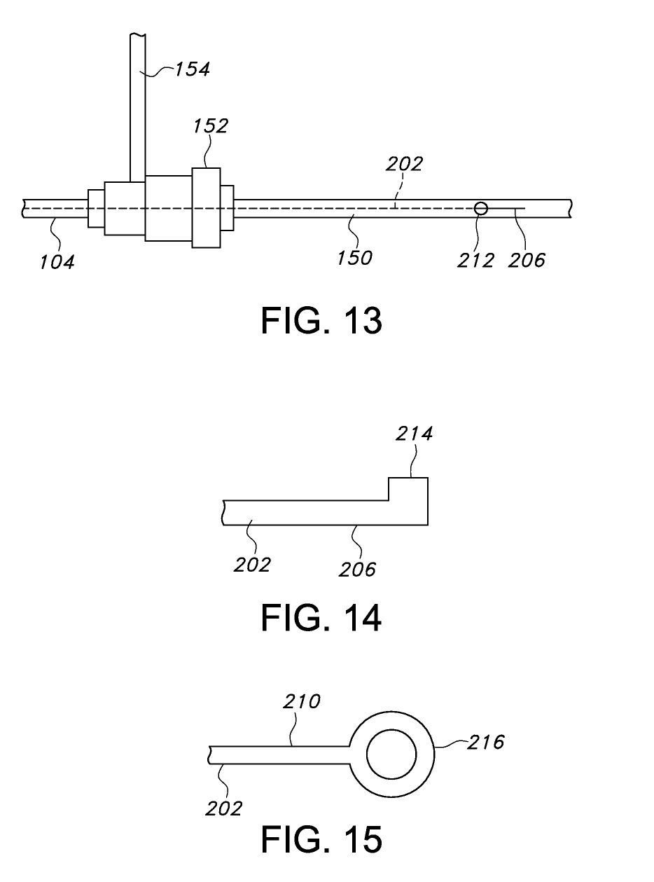

FIGS. 13-15 depict further details of the second guidewire 202. As depicted in FIG. 13, the proximal portion 206 of the second guidewire 202 may exit a proximate portion of the inner tubular member 150 before the proximal handle assembly 170 (not shown in FIG. 13) at a proximal aperture 212 in the inner tubular member 150. A practitioner may manipulate the proximal portion 206 of the second guidewire 202 to advance and/or retract the second guidewire 202. As depicted in FIG. 14 the second guidewire 202 may include a proximal end stop 214 of the second guidewire 202. Such a proximal end stop 214 of the second guidewire 202 may be engaged with the retraction knob or handle 152 for the outer sheath 104 upon retraction of the outer sheath 104 or pulling of the handle 152 by a practitioner. As depicted in FIG. 15 distal end of the guidewire 202 may include a floppy or engagement distal end 216 of the second guidewire 202. While end 216 is depicted as an open circle, any suitable configuration may be used such that the end 216 may be snared by a practitioner. The end 216 may also have enhanced visibility, for example under fluoroscopy, to facilitate snaring by the practitioner.

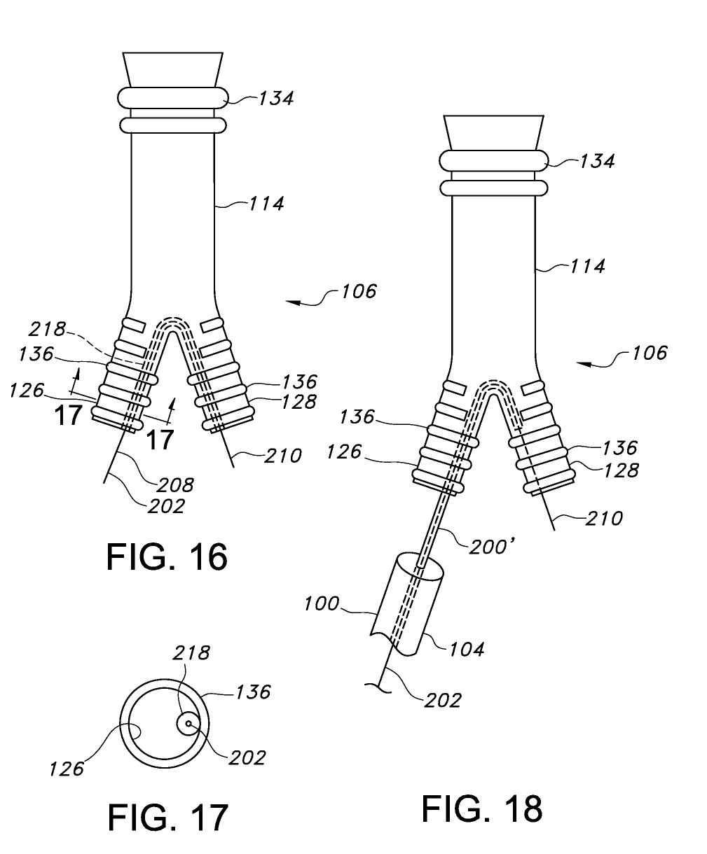

As depicted in FIGS. 16 and 17, the prosthesis 106 of the present invention may include a prosthesis guidewire limen 218. The prosthesis guidewire lumen 218 may extend from the ipsilateral graft leg 126 through the contralateral graft leg 128. While the prosthesis guidewire lumen 218 is depicted as being disposed substantially between the graft ends of the legs 126,128, the present invention is not so limited. For example, the prosthesis guidewire lumen 218 may be disposed within a portion, incusing a substantial portion, of the ipsilateral graft leg 126 and/or the contralateral graft leg 128. The prosthesis guidewire lumen 218 is configured to slidably receive the second guidewire 202. As depicted in FIGS. 16 and 17, the prosthesis guidewire lumen 218 is disposed internally within the prosthesis 106. The prosthesis guidewire lumen 218 may be made from any of the above-described polymeric or biocompatible materials, including those polymeric or biocompatible materials described for the delivery sheath and/or graft.

As depicted in FIG. 18, the delivery system of the present invention may include a second guidewire lumen 200′ which extends or is disposed within the ipsilateral graft leg 126 and within at least a portion of the contralateral graft leg 128. Such a second guidewire lumen 200′ may be retractable by a practitioner through any suitable means, such as a handle (not shown) on the proximal handle assembly 170. The second guidewire lumen 200′ may be biased to have a quiescent curved shape to facilitate placement of the second guidewire lumen 200.

In any of the above described aspects or embodiments of the second guidewire 202 and/or the second guidewire lumen 200,200′; the distal portion 210 of the second guidewire lumen 200 or 200′ may be associated with the release wire 190 (not shown) for the flap 180 at the ipsilateral leg 126. In such a case, the distal portion of the second guidewire lumen 200 or 200′ may be releasably constrained by the release wire 190, where, for example, retraction of the release wire 190 may also release the distal portion of the second guidewire lumen 200 or 200′.

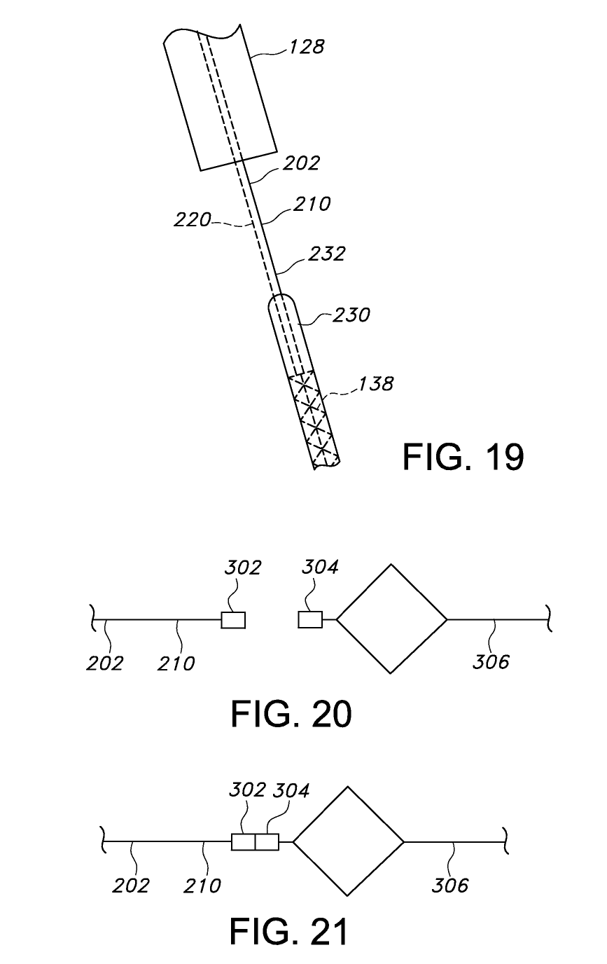

As depicted in FIG. 19, the second guidewire 202 is useful for deploying a second catheter, for example, a catheter 230 for deployment of the contralateral graft extension 138. The end 210 of second guidewire 202 may be snared and withdrawn from the patient's contralateral vascular access site. This wire can then be used to advance a catheter 230 into the contralateral graft leg 128 for deployment of contralateral graft extension 138. Alternatively, after snaring and withdrawing guidewire 202, a third guidewire 220 (shown in phantom or a dashed line) (preferably larger than guidewire 202 and more suitable for catheter 230 advancement and deployment of contralateral graft extension 138) can be inserted alongside guidewire 202 using a small catheter advanced over guidewire 202 having internal lumen diameter large enough to accommodate both the second and third guidewires. The third guidewire 220 is then advanced into contralateral graft leg 128 and graft 114, and the small catheter removed such that catheter 230 can be advanced over the third guidewire and into the contralateral graft leg for deployment of contralateral graft extension 138. Another approach would be to snare end 210 of guidewire 202 from the contralateral side, then while holding the snare tight to guidewire end 210, withdraw guidewire 202 into the contralateral graft leg 128 by pulling handle 204 until the distal end of the snare reaches the graft bifurcation. The snare is then released so that guidewire end 210 can be withdrawn from the snare, and the snare withdrawn from its catheter, leaving its catheter in place with its distal end at the graft bifurcation. A third guidewire is then inserted into the snare catheter and advanced up into the contralateral graft leg such that catheter 230 can be inserted over the third guidewire into the contralateral graft leg for contralateral graft extension 138 deployment.

FIGS. 20 and 21 depict the use of a magnetic snare useful with the present invention. As depicted in FIGS. 20 and 21, the distal portion 210 of the second guidewire 202 a magnetic element 302. A magnetic snare 306 may contain a complimentary magnetic element 304. Such a magnetic element 302 on the end of the distal portion 210 of the second guidewire 202 and a complimentary magnetic element 304 on the end of the magnetic snare 306 may facilitate rapid mating of the second guidewire 202 and the magnetic snare 306, thereby providing cannulated access to the contralateral docking catheter, such as the catheter 230 for deployment of the contralateral graft extension 138. The magnets 302, 304 may be either permanent magnets or electro-magnets, however, permanent magnets would result in a simpler system. Neodymium magnets create the strong magnetic fields and are nearly as strong as electro-magnets. The use of neodymium magnets provides high attractive forces while maintaining for low device profiles. The magnets 302, 304 need to be oriented such that one end of devices 202, 306 has one pole of the magnet and the other end of devices 306, 202 has an opposite pole (north and south or south and north poles of the magnets). As magnetic attractive forces are strongest when the magnetic fields are aligned, it is desirable for the magnets to easily self-align when inserted and in the field of influence of the other magnet. If desired, the attachment of the magnetic element 302 to the guidewire 202 and/or the attachment of the magnetic element 304 to the snare 306 should be flexible. The attractive force should be sufficient to allow the pulling of the mated devices 202, 306 to allow a catheter to be inserted from the contralateral side and advanced up and into the contralateral graft leg 128, after which the mated devices 202, 306 may be removed. The catheter (not shown) for the snare 306 may be configured to easily rotated. When the catheter is rotated, the magnet 304 will sweep out a circle or a larger area for effecting mating of the magnets 302, 304. Alternatively or additionally, the distal portion 210 of the second guidewire 202 may be rotated to facilitate mating of the magnets 302, 304. The second guidewire 202 containing the magnet element 302 may be integrated into the endovascular delivery system 100 or may be provided as an accessory device or application.

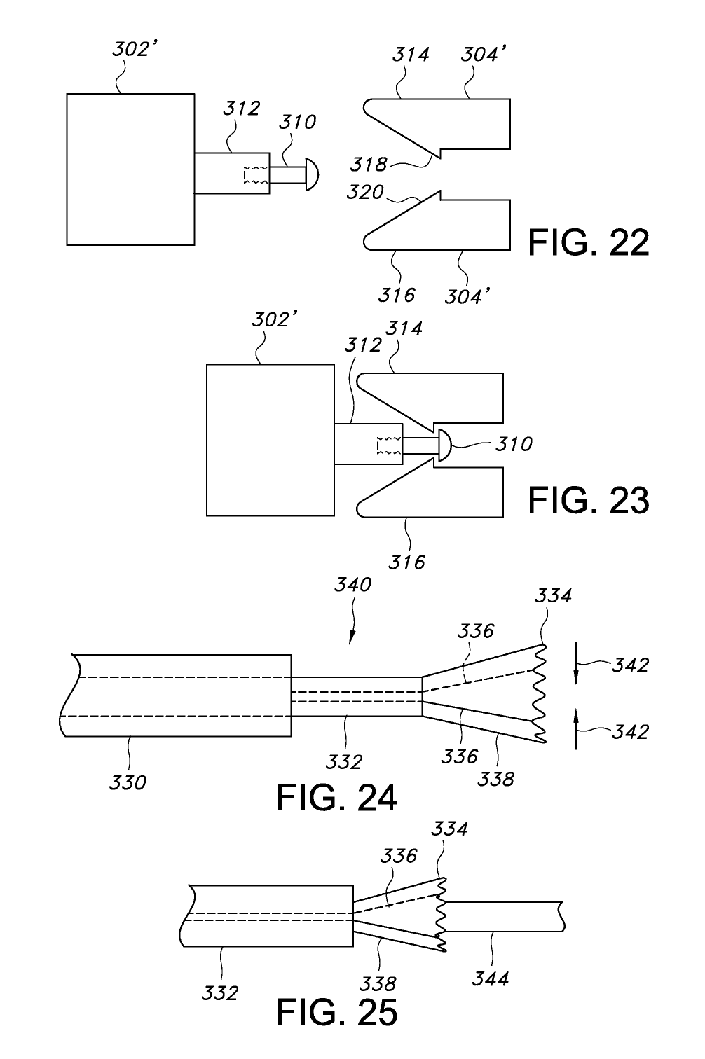

In one embodiment, the magnets 302, 304 may be interlocking, such as interlocking magnets 302′, 304′ as depicted in FIGS. 22 and 23. As depicted in FIGS. 22 and 23, magnet 302′ may include a spring-loaded interlock member 310 (spring not shown). The spring-loaded interlock member 310 may be positioned outward from the magnet 302′ via an interlock post 312. The magnet 304′ may be in the form of two opposed jaws portions 314, 316 having detents 318, 320 for releasably interlocking the spring-loaded interlock member 310. The spring-loaded interlock member 310 may be made of resilient material to allow for the releasably interlocking, or other suitable releasably interlocking configurations may suitable be used. The use the interlocking magnets 302′, 304′ may prevent inadvertent separation of the interlocking magnets 302′, 304′. The present invention is not limited to the use of opposed jaws 314, 316 and other suitable configurations may be used. For example, magnet 304′ may be configured to be a hollow tubular member having detents 318, 320 where the “jaw” portions 314, 316 are flexible enough to permit entry and exit of the spring-loaded interlock member 310. Alternatively or additionally, the spring-loaded interlock member 310 may be configured to permit its entry and exit from the “jaw” portion 314, 316.

FIG. 24 depicts a funnel snaring device 340 useful with the present invention. The funnel snaring device 340 may include a first steerable catheter 330 slidably disposed over a second steerable catheter 332. Disposed on the end of the second steerable catheter 332 is a funnel tube 338. The end of the funnel tube 338 contains a metallic ring 334. The metallic ring 334 may be in the form of a nitinol stent ring. Metallic wires 336 are connected and/or integral with the metallic ring 334. The metallic wires 336 are accessible, either directly or indirectly, to a practitioner for controlling movement of the metallic ring 334 and the funnel tube 338. The funnel tube 338 may be made from any of the above described polymeric materials. One useful material is polytetrafluoroethylene. The metallic ring 334 and the funnel tube 338 are collapsible, as indicated by arrows 342. For example, as depicted in FIG. 25, the metallic ring 334 and the funnel tube 338 are collapsed over a wire 344 to releasably engage the wire 344. The wire 344 may be a wire associated with a snaring device and/or a guidewire associated with a catheter or delivery device or assembly.

In vascular procedures, it may be desirable to cannulate a device (for example to dock another device in the first device, such has iliac limbs or graft extensions 138, 140 into an aortic body, such as graft legs 126, 128 of the main graft body 124 of prosthesis 106. In some cases, such abdominal aortic aneurysms (AAA), an up and over procedure is needed where the access is from one iliac branch and it is desired to come up and over the bifurcation and back down into the other iliac while through a bifurcated AAA repair device. However, sometimes the wire coming down from the bifurcation can point in a direction different from the “other” iliac making it challenging to snare. To make the procedure easier, an interlocking mechanism, such as the funnel snaring device 340, can be employed along with a guiding feature to assist in mating with the interlocking device.

In use, the funnel snaring device 340 may be advanced the ipsilateral iliac artery. Once in the device for repair of AAA, such as prosthesis 106 (not shown), first steerable catheter 330 may be used to bend over the repair device bifurcation. The second steerable catheter 332 may be advanced to the exit of the repair device, and the tip of the second steerable catheter 332 may be manipulated (with rotation if necessary) to point the metallic ring 334 and the funnel tube 338 towards the contralateral iliac ostia. The funnel tube 338 may be manipulated and advanced to make the funnel tube 338 point at the contralateral iliac. A separate interlocking snare wire, such as wire 344, may be advanced up the contralateral iliac. The wire 344 enters the funnel tube 338 until it is interlocked inside the funnel tube 338 and the second steerable catheter 332. The funnel tube 338 may be pulled back to interlock with the interlock snare wire 344. The funnel tube 338 may ten be retracted back and out of the ipsilateral iliac to gain control of the snare wire 344 from either iliac access point, i.e., an access location for the ipsilateral iliac artery and an access location for the contralateral iliac artery. The present invention is not limited to the advancing of the funnel snaring device 340 via the ipsilateral iliac artery, and the funnel snaring device 340 may suitably be advanced via the contralateral iliac artery.

In another embodiment, an accessory, similar to the funnel snaring device 340, may be provided to help access the contra gate in a normal mode (i.e., without going up and over or otherwise having to snare a cross-over wire). The accessory may be used with any of the systems, devices and methods described herein. The accessory (not shown) may be a conical basket/funnel structure, preferably made of nitinol mesh and wire. The accessory would be projected out the end of a catheter once the catheter was in the vicinity of the contra gate. As the funnel is projected out, it opens to a larger diameter at its distal end. This flared, large and fluoro-visible end may be maneuvered so that it butted up against the contra gate. Once it was in apposition to the contra gate, a wire may be advanced from the center of the funnel catheter such that it would enter the contra gate, since it would be contained by the funnel and would not likely escape the funnel and miss its target (i.e., the contra gate). The large funnel is easier to position over the end of the contra gate, in comparison to selecting the gate with a wire using only a two dimensional fluoroscopic view, especially in, for example, a potentially large open aneurysm sac. Moreover, the cannulation aid cone/funnel may have an enhanced steerability. Such enhanced steerability may be as simple as a control mechanism or wire than may permit bending or curving of the cone/funnel. Moreover, the conical basket can be formed in a curve, as can its catheter, so that when the curves are lined up, so that the funnel or basket can have controlled degrees of bending, including relatively large degrees of bending. Further, when they are rotated relative to each other, the funnel/basket may achieve a relatively straight shape.

Another aspect of the present invention is directed to a cross-over catheter, which is designed to provide guidewire access from one side of a patient's vascular system to the other (ipsilateral side to contralateral side). Cross-over techniques are fairly common in vascular procedures. In particular, cross-over techniques are valuable when deploying a bifurcated AAA stent-graft that requires either a pre-delivery cross-over or cannulation step in the procedure. A cross-over technique may be used to gain guidewire access from the patient's opposite side (i.e. contralateral side) retrograde to the aorta, or may also be used for gaining wire access from the ipsilateral to contralateral side (ipsilateral femoral to contralateral side).

Cross-over procedures may be performed with a single lumen catheter in which the distal end of the catheter is in the shape of a shepherd's hook or loop. Such a catheter is soft enough to straighten when a guidewire is placed through the lumen and resilient enough to re-take the shepherd's hook shape once the guidewire is removed from the lumen. A typical cross-over procedure involves: advancing the catheter (over a wire) proximal to the graft or native bifurcation; retracting the guidewire so the distal end of the catheter can re-take the shepherd's hook shape; advancing the wire out of the catheter and down the patient's contralateral side. When using the cross-over technique to gain guidewire access from the contralateral side, the following steps are typically used after the guidewire is crossed-over the bifurcation: the guidewire is snared on the patient's contralateral side; the distal end of the guidewire is pulled out the patient's contralateral side (proximal end of the guidewire remains in the patient's Ipsilateral side); an angiographic catheter is advanced over the cross-over guidewire proximal to the bifurcation; the guidewire from the ipsilateral side is retracted; and a guidewire is advanced from the patient's contralateral side through the angiographic catheter proximal to the bifurcation.

Several factors may make crossing a guidewire over the bifurcation difficult. For example, if too much resistance to advancing the wire is encountered, the guidewire may preferentially straighten the catheter instead of advancing down the contralateral side. Second, the single lumen of the catheter is used with the cross-over guidewire. If the catheter is inadvertently retracted, guidewire access may be lost to both ipsilateral and contralateral sides.

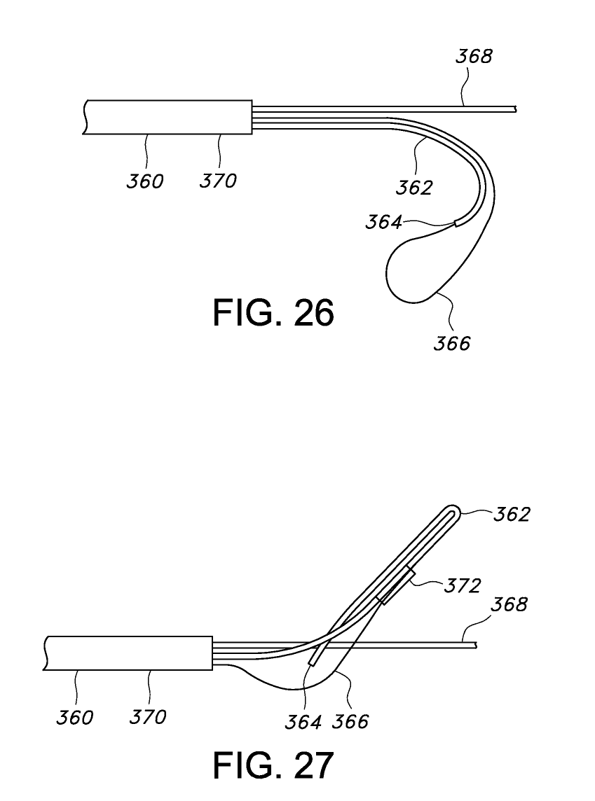

As depicted in FIG. 26, a catheter 360 may be designed to address the two main factors that make crossing a guidewire over the bifurcation difficult. The catheter 360 is constructed with a lumen 362 in the shape of a shepherd's hook. The lumen 362 may be made of a flexible nitinol material, but other materials may also be used. The nitinol material may or may not be shape set to a particular form. A depicted in FIG. 26, the end 364 of the shape set nitinol shepherd's hook lumen 362 is bonded to a steering wire 366. The steering wire 366 is effective at maintaining the shepherd hook's luminal tip position while a guidewire 368 is being advanced through the lumen of the catheter 360. Alternatively, the steering wire 366 may be held fixed by a practitioner while performing the sheath manipulation.

Additionally, the catheter shaft 370 may be constructed of a multi-lumen tubing such that one lumen provides guidewire access for an ipsilateral guidewire 368 while another lumen provides access for the cross-over guidewire (not shown). The catheter may or may not include a protective sheath for ease of use.

The steering wire may be positioned away from the tip 364 of the flexible lumen 362 by bonding it, for example a bonding tube 372, with a sleeve 372 as shown in FIG. 27. This allows a longer straight section of the tip 364 to descend distally into the contralateral leg to ensure passage of a guidewire (not shown) through the leg.

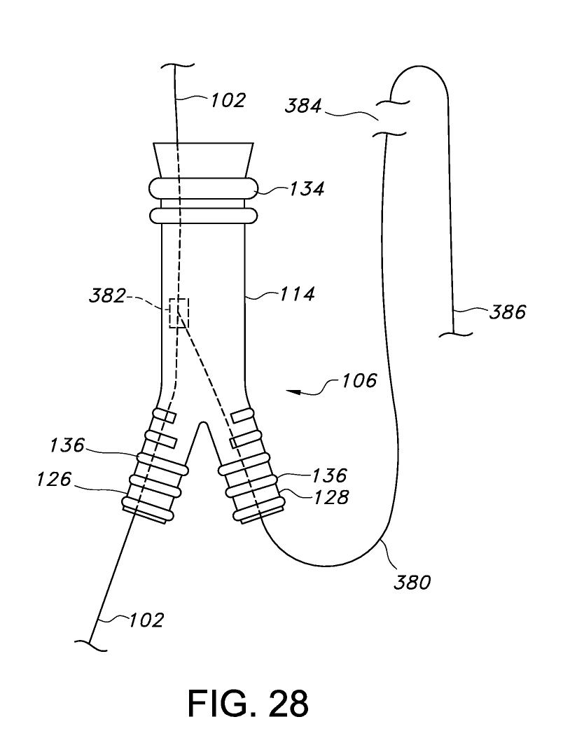

In an another embodiment of the present, method and devices for delivering a modular stent graft by positioning the delivery system in the target vessel through a first access hole with a releasable filament extending out a second access hole. As depicted in FIG. 28, the releasable filament 380 may be attached to the guidewire 102 of the endovascular delivery system 100 or may be otherwise releasably attached to a delivery system, including the endovascular delivery system 100 and routed through the contralateral graft leg 128 of the main graft body 124, and then outside the main graft body 124 to exit the outer sheath 104 (not shown) at the nosecone 120 (not shown, but generally indicated at location 384). The releasable filament 380 may be releasably retained or secured to the guidewire 102 at securement location 382. The securement location 382 may comprise a simple frictional fitting (not shown) between the guidewire 102 and the releasable filament 380. Alternatively, the endovascular delivery system 100 may include a release wire for releasing the releasable filament 380 from the endovascular delivery system 100.

The distal portion 386 of the releasable filament 380 extending from the outer sheath 104 or the distal may be inserted from the patient ipsilateral side (first access hole), over the anatomic bifurcation, and out the contralateral side (second access hole). The releasable filament 380 The releasable filament 380 may then be released and removed through a guide catheter (not shown), and a standard guidewire (not shown) may be routed through the guide catheter.

While various embodiments of the present invention are specifically illustrated and/or described herein, it will be appreciated that modifications and variations of the present invention may be effected by those skilled in the art without departing from the spirit and intended scope of the invention. Further, any of the embodiments or aspects of the invention as described in the claims or in the specification may be used with one and another without limitation. Moreover, while systems, devices and methods have been described generally though introducing main systems and main devices through the ipsilateral iliac artery in a retrograde direction (e.g., femoral access), such main systems and main devices may be introduced through the contralateral iliac artery in a retrograde direction or even through a main or primary artery in a antegrade direction (e.g., subclavian artery access).

What is claimed is:

1. An endovascular delivery system, comprising:
a bifurcated and inflatable prosthesis comprising a main tubular body having an open end and opposed ipsilateral and contralateral legs defining a graft wall therein between, said graft wall having an inner graft wall surface and an outer graft wall surface, and said ipsilateral and contralateral legs having open ends;
an elongate outer tubular sheath having an open lumen and opposed proximal and distal ends with a medial portion therein between, the proximal end of the outer tubular sheath securably disposed to a first handle at a handle assembly;
an elongate inner tubular member having a tubular wall with an open lumen and opposed proximal and distal ends with a proximal portion near the proximal end, a distal portion near the distal end and a medial portion therein between, the inner tubular member having a longitudinal length greater than a longitudinal length of the outer tubular sheath, the inner tubular member being slidably disposed within the open lumen of the outer tubular sheath, the proximal end of the inner tubular member securably disposed to a second handle at the handle assembly; wherein the distal end of the outer tubular sheath being slidably disposed past and beyond the distal end of the inner tubular member to define a prosthesis delivery state and slidably retractable to the medial portion of the inner tubular member to define a prosthesis unsheathed state;
a first elongate guidewire slidably disposed within the inner tubular member and extending from the handle assembly, through the ipsilateral leg of the prosthesis and through the main tubular body of the prosthesis in the prosthesis delivery state;
a second elongate guidewire slidably disposed within the inner tubular member and having a proximal portion extending from the handle assembly or from a proximal portion of the inner tubular member, a medial portion extending through the ipsilateral leg of the prosthesis and a distal portion extending through at least a portion of the contralateral leg of the main tubular body of the prosthesis in the prosthesis delivery state;
whereby the distal portion of the second elongate guidewire is engageable with a catheter to facilitate delivery of a contralateral graft extension within a portion of the contralateral leg of the main tubular body of the prosthesis in the prosthesis unsheathed state upon proximally retracting the second elongate guidewire.

2. The endovascular delivery system of claim 1, wherein and said main tubular body and said ipsilateral and contralateral legs comprise inflatable channels.

3. The endovascular delivery system of claim 1, wherein the distal portion of the second elongate guidewire is disposed beyond the open end of the contralateral leg of the main tubular body in the prosthesis unsheathed state.

4. The endovascular delivery system of claim 1,
wherein the prosthesis further comprises a prosthesis guidewire lumen extending from at least a portion of the inner graft wall surface of the ipsilateral leg of the prosthesis and extending to at least a portion of the inner graft wall surface of the contralateral leg of the prosthesis; and
wherein at least a portion of the medial portion of the second elongate guidewire and at least a portion of the distal portion of the second elongate guidewire are disposed within prosthesis guidewire lumen of the prosthesis.

5. The endovascular delivery system of claim 1,
further comprising a delivery system second guidewire lumen extending over at least a portion of the medial portion of the second elongate guidewire and over at least a portion of the distal portion of the second elongate guidewire and;
wherein in the prosthesis delivery state the delivery system second guide wire lumen extends from at least a portion of the ipsilateral leg of the prosthesis to a portion of the contralateral leg of the prosthesis.

6. The endovascular delivery system of claim 1,
further comprising a third handle at the handle assembly;
wherein the proximal portion of the second elongate guidewire is secured to the third handle to proximally retract the second elongate guidewire.

7. The endovascular delivery system of claim 1,
wherein the proximal portion of the second elongate guidewire extends through a wall of the proximal portion of the inner tubular member; and
wherein retraction of the outer tubular member engages the proximal portion of the second elongate guidewire to proximally retract the second elongate guidewire.

8. The endovascular delivery system of claim 1, wherein the prosthesis comprises non-textile polymeric material.

9. The endovascular delivery system of claim 8, wherein the non-textile polymeric material of the prosthesis comprises extruded polytetrafluoroethylene.

10. The endovascular delivery system of claim 9, wherein said extruded polytetrafluoroethylene is non-porous polytetrafluoroethylene.

11. The endovascular delivery system of claim 1, wherein the prosthesis further comprises a metallic expandable member securably disposed at or near the open end of the main tubular body of said prosthesis.

12. A method for delivering a bifurcated prosthesis, comprising:
providing the endovascular delivery system of claim 1;
advancing the endovascular delivery system through a first branched artery and into an aneurysm in a main artery;
retracting the outer sheath to deploy the prosthesis so the proximal end of the main tubular body of the prosthesis is disposed beyond the aneurysm and so that the ipsilateral and contralateral legs are disposed within the aneurysm;
advancing a catheter through a second branched artery;
engaging the catheter with the distal portion of the second elongate guidewire;
retracting the second elongate guidewire proximally to advance the catheter within a portion of the contralateral leg of the prosthesis;
disengaging the second elongate guidewire and the catheter from one and the other; and
further retracting the second elongate guidewire at least partially through the ipsilateral leg of the prosthesis.

13. The method of claim 12 further comprising:
maintaining the first elongate guidewire through the ipsilateral leg and the main tubular body of the prosthesis while retracting the second elongate guidewire through the ipsilateral leg of the prosthesis.

14. The method of claim 12 further comprising:
deploying a contralateral graft extension having opposed proximal and distal open ends contained within a catheter so that the proximal end of the contralateral graft extension is disposed within a portion of the contralateral leg of the main tubular body of the prosthesis and so that the distal end of the contralateral graft extension is disposed distally of the aneurysm and within a portion of the second branched artery.

15. The method of claim 12 further comprising:
advancing a second catheter through the first branched artery along the first elongate guidewire;
deploying a ipsilateral graft extension having opposed proximal and distal open ends contained within a second catheter so that the proximal end of the ipsilateral graft extension is disposed within a portion of the ipsilateral leg of the main tubular body of the prosthesis and so that the distal end of the ipsilateral graft extension is disposed distally of the aneurysm and within a portion of the first branched artery.

16. A method for delivering a bifurcated prosthesis, comprising:
providing the endovascular delivery system comprising:
a bifurcated and inflatable prosthesis comprising a main tubular body having an open end and opposed ipsilateral and contralateral legs defining a graft wall therein between;
an elongate outer tubular sheath having an open lumen and opposed proximal and distal ends with a medial portion therein between;
an elongate inner tubular member having a tubular wall with an open lumen and opposed proximal and distal ends with a proximal portion near the proximal end, a distal portion near the distal end and a medial portion therein between, the inner tubular member having a longitudinal length greater than a longitudinal length of the outer tubular sheath, the inner tubular member being slidably disposed within the open lumen of the outer tubular sheath, wherein the distal end of the outer tubular sheath being slidably disposed past and beyond the distal end of the inner tubular member to define a prosthesis delivery state and slidably retractable to the medial portion of the inner tubular member to define a prosthesis unsheathed state;
a first elongate guidewire slidably disposed within the inner tubular member and extending through the ipsilateral leg of the prosthesis and through the main tubular body of the prosthesis in the prosthesis delivery state;
a second elongate guidewire slidably disposed within the inner tubular member and extending through the ipsilateral leg of the prosthesis and having a distal portion extending through at least a portion of the contralateral leg of the main tubular body of the prosthesis in the prosthesis delivery state;
advancing the endovascular delivery system through a first branched artery and into an aneurysm in a main artery;
retracting the outer sheath to deploy the prosthesis so the proximal end of the main tubular body of the prosthesis is disposed beyond the aneurysm and so that the ipsilateral and contralateral legs are disposed within the aneurysm;
advancing a catheter through a second branched artery;
engaging the catheter with the distal portion of the second elongate guidewire;
retracting the second elongate guidewire proximally to advance the catheter within a portion of the contralateral leg of the prosthesis;
disengaging the second elongate guidewire and the catheter from one and the other; and
further retracting the second elongate guidewire at least partially through the ipsilateral leg of the prosthesis.

17. The method of claim 16 further comprising:
maintaining the first elongate guidewire through the ipsilateral leg and the main tubular body of the prosthesis while retracting the second elongate guidewire through the ipsilateral leg of the prosthesis.

18. The method of claim 16 further comprising:
deploying a contralateral graft extension having opposed proximal and distal open ends contained within a catheter so that the proximal end of the contralateral graft extension is disposed within a portion of the contralateral leg of the main tubular body of the prosthesis and so that the distal end of the contralateral graft extension is disposed distally of the aneurysm and within a portion of the second branched artery.

19. The method of claim 16 further comprising:
advancing a second catheter through the first branched artery along the first elongate guidewire;
deploying a ipsilateral graft extension having opposed proximal and distal open ends contained within a second catheter so that the proximal end of the ipsilateral graft extension is disposed within a portion of the ipsilateral leg of the main tubular body of the prosthesis and so that the distal end of the ipsilateral graft extension is disposed distally of the aneurysm and within a portion of the first branched artery.

20. An endovascular prosthesis, comprising:
a bifurcated and inflatable prosthesis comprising:
a main tubular body having an open end and opposed ipsilateral and contralateral legs defining a graft wall therein between, said graft wall having an inner graft wall surface and an outer graft wall surface, and said ipsilateral and contralateral legs having open ends; and
a prosthesis guidewire lumen extending along at least a portion of the inner graft wall surface of the ipsilateral leg of the prosthesis and extending along at least a portion of the inner graft wall surface of the contralateral leg of the prosthesis;
wherein the prosthesis guidewire lumen is configured to receive a guidewire from a delivery system.

21. The endovascular prosthesis of claim 20, wherein said main tubular body and said ipsilateral and contralateral legs comprise inflatable channels.

22. The endovascular prosthesis of claim 20, wherein the prosthesis comprises non-textile polymeric material.

23. The endovascular prosthesis of claim 22, wherein the non-textile polymeric material of the prosthesis comprises extruded polytetrafluoroethylene.

24. The endovascular prosthesis of claim 23, wherein said extruded polytetrafluoroethylene is non-porous polytetrafluoroethylene.

25. The endovascular prosthesis of claim 20, wherein the prosthesis further comprises a metallic expandable member securably disposed at or near the open end of the main tubular body of said prosthesis.
